(19) United States
(12) Patent Application Publication
(10) Pub. No.: US 2010/0105612 A1
(43) Pub. Date: Apr. 29, 2010

(54) AGENT CONTAINING FUSED PROTEIN OF SOLUBLE RANKL WITH EPITOPE TAG

(75) Inventors: Yoshiya Tomimori, Shiga (JP); Hisataka Yasuda, Shiga (JP)

Correspondence Address: HAMRE, SCHUMANN, MUELLER & LARSON, P.C., P.O. BOX 2902, MINNEAPOLIS, MN, 55402-0902, US

(73) Assignee: ORIENTAL YEAST CO., LTD., Tokyo (JP)

(21) Appl. No.: 12/445,033

(22) PCT Filed: Jul. 5, 2007
(86) PCT No.: PCT/JP2007/063869

§ 371 Date: Apr. 9, 2009

(30) Foreign Application Priority Data
Oct. 11, 2006 (JP) 2006-278052

(51) Int. Cl. A61K 38/16; C07K 14/00; C12N 9/96; C12N 5/00; A61P 43/00

(52) U.S. Cl. 514/12; 530/402; 435/188; 435/377

(57) ABSTRACT

The present invention provides a reagent containing a fused protein of RANKL with an epitope tag that has improved effects of differentiating and activating osteoclasts and improved preservation stability compared with the case of using RANKL alone, and an agent for differentiating and activating osteoclasts that can be used in vitro or in vivo, containing, as an active ingredient, a fused protein of soluble RANKL with an epitope tag peptide.

### TECHNICAL FIELD

The present invention relates to an agent containing a fused protein of soluble RANKL with an epitope tag, such protein being capable of inducing osteoclast differentiation and activation.

### BACKGROUND ART

Osteoclasts, which control osteolysis, are large multinucleated cells derived from hematopoietic cells that differentiate into monocytes/macrophages. Differentiation and maturation of osteoclast precursor cells into osteoclasts are controlled by osteoblasts/stromal cells on the bone surface. An osteoclast differentiation factor (RANKL; receptor activator of NF-κB ligand) is a membrane-bound protein belonging to the family of tumor necrosis factors (TNFs) guided by bone resorption factors onto osteoblasts/stromal cells, and it is essential for osteoclast differentiation and maturation (Non-Patent Documents 1 and 2). It has been known that RANKL is partially cleaved by metalloprotease in an extracellular region so as to result in soluble RANKL. In practice, soluble RANKL is known to induce in vitro differentiation of macrophage precursor cells into osteoclasts when coexisting with M-CSF. However, it cannot be said that soluble RANKL products, including those that are commercially available, have strong levels of bioactivity. Therefore, even when osteoclasts are formed by cell culture with the use of myelocytes, spleen cells, precursor cells in the peripheral blood, a macrophage cell line, or the like, a number of very large osteoclasts cannot be readily obtained.

Meanwhile, in order to increase the amount of recombinant protein produced or to facilitate recombinant protein purification, a variety of proteins and peptides have been used, which can also be used as tags. Such proteins and peptides include glutathione-S-transferase (GST) (see Non-Patent Document 3), histidine tag (His tag)/thioredoxin (TRX) (see Patent Documents 1 and 2 and Non-Patent Documents 4 and 5), flag tag (FLAG) (see Patent Documents 3 and 4), Myc tag (see Non-Patent Document 6), V5 tag (see Non-Patent Document 7), Xpress tag, and an immunoglobulin Fc region. These proteins and peptides have been used for the above purposes; however, they have not been used for the purpose of increasing the activity of fused protein.
Patent Document 1: U.S. Pat. No. 5,270,181
Patent Document 2: U.S. Pat. No. 5,292,646
Patent Document 3: JP Patent No. 1983150
Patent Document 4: JP Patent No. 2665359
Non-Patent Document 1: Yasuda et al., Proc Natl Acad Sci USA 95: 3597, 1998
Non-Patent Document 2: Lacey et al., Cell 93: 165, 1998
Non-Patent Document 3: Kaelin et al., Cell 70: 351, 1992
Non-Patent Document 4: La Vallie et al., Bio/Technology 11: 187, 1993
Non-Patent Document 5: Lu et al., J Biol Chem 271: 5059, 1996
Non-Patent Document 6: Evans et al., Mol Cell Biol 5, 3610, 1985
Non-Patent Document 7: Southern et al., J Gen Virol 72, 1551, 1991

### DISCLOSURE OF THE INVENTION

It is an object of the present invention to provide a reagent containing a fused protein of RANKL with an epitope tag that has improved effects of differentiating and activating osteoclasts compared with the case of using RANKL alone.

The present inventors have found that the activity of soluble RANKL can be enhanced in vitro and in vivo by fusing soluble RANKL with a different type of protein or peptide used as a tag. This had led to the completion of the present invention.

Specifically, the present invention is described as follows.

[1] An agent for differentiating and activating osteoclasts, containing, as an active ingredient, a fused protein of soluble RANKL with an epitope tag in which the differentiating and activating function of soluble RANKL is improved.
[2] The agent for differentiating and activating osteoclasts according to [1], which is used in vitro.
[3] The agent for differentiating and activating osteoclasts according to [1], which is used in vivo.
[4] The agent for differentiating and activating osteoclasts according to any one of [1] to [3], which has improved preservation stability.
[5] The agent for differentiating and activating osteoclasts according to any one of [1] to [4], which has interspecies cross-reactivity.
[6] An agent for inducing osteopenia in an animal, containing, as an active ingredient, a fused protein of soluble RANKL with an epitope tag in which the differentiating and activating function of soluble RANKL is improved.
[7] The agent for inducing osteopenia in an animal according to [6], which has interspecies cross-reactivity.
[8] An agent for producing an osteopenia animal model, containing, as an active ingredient, a fused protein of soluble RANKL with an epitope tag in which the differentiating and activating function of soluble RANKL is improved.
[9] The agent for producing an osteopenia animal model according to [8], which has interspecies cross-reactivity.
[10] The agent for differentiating and activating osteoclasts according to any one of [1] to [5], wherein the epitope tag is glutathione-S-transferase.
[11] The agent for inducing osteopenia in an animal according to [6] or [7], wherein the epitope tag is glutathione-S-transferase.
[12] The agent for producing an osteopenia animal model according to [8] or [9], wherein the epitope tag is glutathione-S-transferase.
[13] A method of differentiating and forming osteoclasts from myelocytes, spleen cells, or peripheral blood cells, comprising culturing myelocytes, spleen cells, or peripheral blood cells collected from an animal in the presence of the agent for differentiating and activating osteoclasts according to [1] to [5].
[14] A method of inducing osteopenia in a non-human animal, comprising administering the agent for differentiating and activating osteoclasts according to any one of [1] to [5] to the non-human animal.
[15] A composition selected from the group consisting of an agent for differentiating lymphocytes, a dendritic cell activator, an agent for differentiating mammary gland epithelial cells, and an agent for forming lymph nodes, which contains, as an active ingredient, a fused protein of soluble RANKL with an epitope tag in which at least one function of soluble RANKL selected from the group consisting of a lymphocyte differentiating function, a dendritic cell activating function, a mammary gland epithelial cell differentiating function, and a lymph node forming function has been improved.
[16] The composition according to [15], which has interspecies cross-reactivity.
[17] The composition according to [15] or [16], wherein the epitope tag is glutathione-S-transferase.

This description includes part or all of the contents as disclosed in the description and/or drawings of Japanese Patent Application No. 2006-278052, which is a priority document of the present application.

### BRIEF DESCRIPTION OF THE DRAWINGS

FIGS. 1A and 1B show images of osteoclast differentiation and formation induced by a fused protein of soluble RANKL with an epitope tag.

FIG. 2 is a graph showing TRAP activity (absorbance) representing the degree of osteoclast differentiation and formation induced by a fused protein of soluble RANKL with an epitope tag.

FIGS. 3A and 3B are graphs each showing effects exhibited by a fused protein of soluble RANKL with an epitope tag upon osteoclasts in terms of life-extension (3A) and activation (3B).

FIG. 4 is a graph showing fluctuations in the serum calcium concentration in a case in which a fused protein of soluble RANKL with an epitope tag was administered to mice.

FIG. 5 is a graph showing fluctuations in the serum CTx concentration in a case in which a fused protein of soluble RANKL with an epitope tag was administered to mice.

FIG. 6 is a graph showing fluctuations in the serum TRAP-5b concentration in a case in which a fused protein of soluble RANKL with an epitope tag was administered to mice.

FIG. 7 is a graph showing fluctuations in the serum osteocalcin concentration in a case in which a fused protein of soluble RANKL with an epitope tag was administered to mice.

FIG. 8 is a graph showing fluctuations in the serum ALP concentration in a case in which a fused protein of soluble RANKL with an epitope tag was administered to mice.

FIGS. 9A and 9B are graphs each showing fluctuations in femur bone density in a case in which a fused protein of soluble RANKL with an epitope tag was administered to mice.

FIG. 10A is a graph showing TRAP activity (absorbance) representing the degree of osteoclast differentiation and formation induced by a fused protein of soluble RANKL with an epitope tag.

FIG. 10B shows images of osteoclast differentiation and formation induced by a fused protein of soluble RANKL with an epitope tag.

FIGS. 11A and 11B are graphs each showing preservation stability of a fused protein of soluble RANKL with an epitope tag. In FIG. 11A, the TRAP activity (absorbance) represents the osteoclast differentiation activity before preservation. In FIG. 11B, the TRAP activity (absorbance) represents the osteoclast differentiation activity after preservation for 2 months.

FIG. 12 is a chart showing the cross-reactivity of a fused protein of soluble RANKL with an epitope tag. RANKL1 denotes mouse soluble RANKL (produced by Peprotech) and RANKL2 denotes mouse soluble RANKL (produced by R&D).

FIG. 13 shows images of osteoclast formation induced by soluble RANKL and GST-RANKL.

### BEST MODE FOR CARRYING OUT THE INVENTION

Hereinafter, the present invention is described in greater detail.

The agent for differentiating and activating osteoclasts, the agent for inducing osteopenia in an animal, and the agent for producing an osteopenia animal model of the present invention each contain, as an active ingredient, a fused protein of soluble RANKL (sRANKL) with an epitope tag.

RANKL (receptor activator of NF-κB ligand) serves as a ligand for RANK (receptor activator of NF-κB), which is a TNF super family member, and RANKL is a type 2 transmembrane protein having an intracellular domain (a domain comprising amino acids at positions 1 to 48 from the N-terminal of RANK), a transmembrane domain, and an extracellular domain (JP Patent Publication (Kohyo) No. 2002-509430 A and WO98/46644 (JP Patent No. 3523650)). In the extracellular domain, a domain comprising amino acids at position 152 from the N-terminal and the following positions is a TNF ligand family homologous domain. Soluble RANKL does not contain an intracellular domain.

Soluble RANKL includes a soluble RANKL derivative and a soluble RANKL analog. The animal origin of soluble RANKL is not limited, and thus RANKL derived from any animal species, such as human-derived RANKL, mouse-derived RANKL, or rat-derived RANKL, can be used. The full-length nucleotide sequence and the amino acid sequence of human-derived RANKL are represented by SEQ ID NOS: 1 and 2, respectively. A soluble RANKL derivative or a soluble RANKL analog includes a protein comprising a partial sequence of the amino acid sequence of RANKL and having the RANKL activity, such as a truncated protein of RANKL. Preferably, a soluble RANKL derivative comprises a TNF ligand family homologous domain starting from an amino acid at position 152 in the amino acid sequence represented by SEQ ID NO: 2. Examples of a soluble RANKL derivative include a protein having an amino acid sequence comprising amino acids at positions 127 to 317, a protein having an amino acid sequence comprising amino acids at positions 140 to 317, and a protein having an amino acid sequence comprising amino acids at positions 159 to 317. Another example thereof is an RANKL derivative derived from a non-human animal, which has an amino acid sequence corresponding to one of the above partial amino acid sequences of human RANKL. Further, examples of a soluble RANKL derivative or a soluble RANKL analog include: a protein having RANKL activity and comprising an amino acid sequence derived from the amino acid sequence represented by SEQ ID NO: 2 by deletion, substitution, or addition of one or several amino acid(s); and a protein having RANKL activity and comprising an amino acid sequence derived from the amino acid sequence of one of the above proteins each comprising a partial amino acid sequence of RANKL by deletion, substitution, or addition of one or several amino acid(s). Herein, the term “one or several” means 1 to 9, preferably 1 to 5, and more preferably 1 or 2.

An epitope tag that forms a fused protein together with soluble RANKL can be a protein or peptide having a sequence capable of binding to a specific compound such as an antibody. In general, an epitope tag is used for fused protein purification. However, in the present invention, an epitope tag has a function of increasing the activity of soluble RANKL. In addition, such an epitope tag has a function of increasing preservation stability in a soluble RANKL solution during cryopreservation.

Examples of an epitope tag include, but are not limited to: glutathione-S-transferase (GST); polyhistidine comprising 2 to 12, preferably 4 or more, more preferably 4 to 7, and further preferably 5 or 6 histidines; FLAG tag (amino acid sequence DYKDDDDK; SEQ ID NO: 3); Myc tag (amino acid sequence EQKLISEEDL; SEQ ID NO: 4); V5 tag (amino acid sequence GKPIPNPLLGLDST; SEQ ID NO: 5); Xpress tag; HQ tag (amino acid sequence HQHQHQ; SEQ ID NO: 6); HA tag (amino acid sequence YPYDVPDYA; SEQ ID NO: 7); AU1 tag (amino acid sequence DTYRYI; SEQ ID NO: 8); T7 tag (amino acid sequence MASMTGGQQMG; SEQ ID NO: 9); VSV-G tag (amino acid sequence YTDIEMNRLGK; SEQ ID NO: 10); DDDDK tag (amino acid sequence DDDDK; SEQ ID NO: 11); S tag (amino acid sequence KETAAAKFERQHIDSC; SEQ ID NO: 12); CruzTag09 (amino acid sequence MKAEFRRQESDR; SEQ ID NO: 13); CruzTag22 (amino acid sequence MRDALDRLDRLA; SEQ ID NO: 14); CruzTag41 (amino acid sequence MKDGEEYSRAFR; SEQ ID NO: 15); Glu-Glu tag (amino acid sequence EEEEYMPME; SEQ ID NO: 16); Ha.11 tag (amino acid sequence CTPTDVPDYASL; SEQ ID NO: 17); KT3 tag (amino acid sequence PPEPET; SEQ ID NO: 18); thioredoxin; a maltose binding protein (MBP); an immunoglobulin Fc region; and β-galactosidase. Of these, glutathione-S-transferase is preferable.

A fused protein of soluble RANKL with an epitope tag can be obtained by ligating the genes encoding the respective components to each other and causing the expression of the resultant. Fusion of the gene encoding RANKL with the gene encoding an epitope tag can be carried out by a conventional gene recombination method with the introduction of appropriate restriction sites. In such case, it is necessary to exclude a stop codon between the genes to be fused. The distance between the genes to be fused is not limited, and a linker may be contained therebetween. In addition, it is necessary to allow the open reading frames of the two genes to overlap each other. The above epitope tag can be fused either on the N-terminal side or on the C-terminal side of the amino acid sequence of RANKL.

The nucleotide sequence of DNA encoding a fused protein of GST with a protein having an amino acid sequence comprising amino acids at positions 127 to 317 of the amino acid sequence of RANKL and the amino acid sequence of the fused protein are represented by SEQ ID NOS: 19 and 20, respectively. The nucleotide sequence of DNA encoding a fused protein of GST with a protein having an amino acid sequence comprising amino acids at positions 140 to 317 of the amino acid sequence of RANKL and the amino acid sequence of the fused protein are represented by SEQ ID NOS: 21 and 22, respectively. In addition, the nucleotide sequence of DNA encoding a fused protein of GST with a protein having an amino acid sequence comprising amino acids at positions 159 to 317 of the amino acid sequence of RANKL and the amino acid sequence of the fused protein are represented by SEQ ID NOS: 23 and 24, respectively.

The thus produced fused gene is incorporated into an appropriate available expression vector so as to be expressed therein such that a fused protein of interest can be recovered and purified. In addition, the gene can be expressed also in a cell-free system.

Any vector can be used as a vector as long as the vector can be replicated in host cells such as plasmids, phages, and viruses. A vector comprises a replication origin, a selection marker, and a promoter. It may further comprise an enhancer, a transcription termination sequence (terminator), a ribosome binding site, a polyadenylation signal, and the like, according to need. Alternatively, a vector into which a gene encoding an epitope tag such as glutathione-S-transferase has been incorporated in a preliminary step can be used.

DNA can be introduced into a vector by a conventionally known method. Desirably, such a vector comprises: a polylinker containing different restriction sites; or a single restriction site. A specific restriction site in a vector is cleaved with a specific restriction enzyme and DNA can be inserted into the cleavage site. An expression vector containing a fused gene is used for transformation of an appropriate host cell such that a fused protein encoded by the fused gene can be expressed and produced in the host cell.

Examples of a host cell include: bacterial cells of Escherichia coli, Streptomyces, Bacillus subtilis, and the like; fungal cells; bakers' yeast cells; yeast cells; insect cells; and mammalian cells.

Transformation can be carried out by a conventionally known method such as the calcium chloride method, the calcium phosphate method, DEAE-dextran mediated transfection, electroporation, lipofection, or the like.

The obtained recombinant fusion protein can be purified by a variety of purification methods. For instance, ammonium sulfate precipitation, gel filtration, ion-exchange chromatography, affinity chromatography, and the like can be used alone or in combination according to need. In a case in which an expression product is expressed as a fused protein comprising GST or the like, purification can be carried out based on the characteristics of a protein or peptide fused with a protein of interest. For instance, when a fused protein comprising GST is expressed, GST has an affinity to glutathione and therefore the fused protein can be efficiently purified by affinity chromatography with the use of a column containing glutathione-bound carriers. Also, when a fused protein comprising a histidine tag is expressed, such a protein having a histidine tag binds to a chelate column and therefore the fused protein can be purified with the use of a chelate column. Further, a fused protein comprising an arbitrary epitope tag can be purified by affinity chromatography with the use of an antibody that recognizes an epitope of the epitope tag.

The activity of soluble RANKL can be enhanced not only in vitro but also in vivo in the case of the fused protein of soluble RANKL with an epitope tag of the present invention. Herein, the term “activity of soluble RANKL” refers to in vitro activity of forming osteoclasts by cell culture with the use of myelocytes, spleen cells, precursor cells in the peripheral blood, a macrophage cell line, or the like, or in vitro activity of promoting life extension or bone resorption potency of purified osteoclasts. Also, the activity refers to in vivo activity of increasing the number of osteoclasts so as to activate osteoclasts for promotion of bone resorption. Specifically, increases or decreases in the activity of RANKL can be found based on increases in the number of osteoclasts on the bone surface and in the osteoclast surface area, decreases in bone density and in bone mass, increases in serum bone resorption markers (e.g., calcium, a degraded collagen product (CTx), and tartrate-resistant acid phosphatase (TRAP-5b)), and the like.

The fused protein of soluble RANKL with an epitope tag peptide of the present invention induces in vitro and in vivo osteoclast differentiation, and it further activates and promotes the bone resorption activity of matured osteoclasts. Soluble RANKL alone, to which an epitope tag has not been bound, can also exhibit such effects. However, the fused protein of soluble RANKL with an epitope tag peptide of the present invention has improved osteoclast differentiation and activation potency compared with the case of using RANKL alone. The osteoclast differentiation and activation potency of the fused protein of soluble RANKL with an epitope tag peptide is significantly improved compared with the case of using soluble RANKL alone. For instance, in a case in which such fused protein is added in vitro to myelocytes or spleen cells derived from an animal (e.g., a human, a mouse, or a rat), RAW cells, which are mouse macrophage-like cells, or the like, the number and the size of formed osteoclasts increases to a greater extent than in the case of the addition of soluble RANKL alone. In addition, in the above case, the life extension of isolated matured osteoclasts and the improvement in bone resorption potency of osteoclasts can be achieved to a greater extent than in the case of the addition of soluble RANKL alone. Further, the potency inherent in RANKL is enhanced in the fused protein of soluble RANKL with an epitope tag peptide, and thus it exhibits effects of differentiating and activating osteoclasts to an extent that cannot be achieved with the addition of RANKL alone.

When used in vitro, the fused protein of soluble RANKL with an epitope tag peptide is added at a concentration of 0.1 nM or more, preferably 0.5 nM or more, more preferably 1 nM or more, further preferably 2 nM or more, even further preferably 2.5 nM or more, and even further preferably 5 nM or more.

In addition, M-CSF may be added when the fused protein of soluble RANKL with an epitope tag is used in vitro to induce osteoclast differentiation and activation.

As described above, the fused protein of soluble RANKL with an epitope tag peptide can be used in vitro and in vivo as an agent for differentiating and activating osteoclasts and thus it can be preferably used for studies of bone metabolism in animals and the like.

In addition, when the fused protein of soluble RANKL with an epitope tag is administered in vivo to an animal, the fused protein induces osteoclast differentiation in the body of the animal and further activates and promotes the bone resorption activity of matured osteoclasts. Further, increases in the number of osteoclasts on the bone surface and in the osteoclast surface area, decreases in the bone density and in the bone mass, and increases in serum bone resorption markers are observed in the animal. Such effects are more significantly improved when the fused protein of soluble RANKL with an epitope tag is administered to an animal than when soluble RANKL alone is administered to the same.

Animals to which the fused protein of soluble RANKL with an epitope tag is administered are not limited, and thus it can be administered to all animals such as humans, monkeys, mice, and rats. The fused protein of soluble RANKL with an epitope tag acts on animals irrespective of species differences. Specifically, for instance, a fused protein of human-derived soluble RANKL with an epitope tag acts on RANKs derived from the other animals such as mice, monkeys, and rats, and such a fused protein also exhibits osteoclast induction activity in the other animal species. According to the present invention, when a fused protein of soluble RANKL with an epitope tag acts on an animal irrespective of species differences, it can be said that such a fused protein has the cross-reactivity, interspecies cross-reactivity, or effects based on interspecies cross-reactivity.

M-CSF also may be added also in a case in which the above fused protein of soluble RANKL with an epitope tag is administered to an animal.

The amount of the fused protein of soluble RANKL with an epitope tag to be administered to an animal is not limited, and it can be adequately determined depending on animal species. For instance, the fused protein can be administered to mice in an amount of 10 nmol to 5000 nmol and preferably 50 nmol to 1000 nmol per individual mouse. The administration route is not limited and thus the fused protein can be administered in the form of an intravenous injection, an intraperitoneal injection, a subcutaneous injection, a muscular injection, a suppository, an ophthalmic preparation, or the like.

As described above, the fused protein of soluble RANKL with an epitope tag can be used as an agent for inducing osteopenia. Further, an animal with a decrease in bone mass can be used as an osteopenia animal model for screening for a therapeutic or prophilaxic agent for bone diseases characterized by decreases in bone density and bone mass. Examples of bone diseases characterized by decreases in bone density and bone mass include osteoporosis and osteopenia.

In addition, the activity of RANKL is not limited to osteoclast differentiation and activation, and it also includes activity of promoting lymphocyte differentiation, dendritic cell activation, mammary gland epithelial cell differentiation, lymph node formation, and the like. Thus, the fused protein can also be used for a method of inducing promotion of such effects. That is, the present invention encompasses a composition selected from the group consisting of an agent for differentiating lymphocytes, a dendritic cell activator, an agent for differentiating mammary gland epithelial cells, and an agent for forming lymph nodes, which contains, as an active ingredient, a fused protein of soluble RANKL with an epitope tag in which at least one function of soluble RANKL selected from the group consisting of a lymphocyte differentiating function, a dendritic cell activating function, a mammary gland epithelial cell differentiating function, and a lymph node forming function has been improved.

The agent for differentiating and activating osteoclasts and the other compositions of the present invention may contain a carrier, a diluent, or an excipient which are generally used in the field of drug formulation. Examples of a carrier or an excipient that can be used for tablets include lactose and magnesium stearate. Examples of an injectable aqueous liquid that can be used include physiological saline, glucose, and isotonic solutions containing different adjuvants, which can be used in combination with an appropriate solubilizing adjuvant such as polyalcohol (e.g., alcohol or propylene glycol) or a nonion surfactant activator. Examples of an oily liquid that can be used include sesame oil and soybean oil, which can be used in combination with a solubilizing adjuvant such as benzyl benzoate or benzyl alcohol.

The present invention is hereafter described in greater detail with reference to the following examples, although the technical scope of the present invention is not limited thereto.

### Example 1

### Confirmation of In Vitro Effects of GST-RANKL

### Preparation of GST-RANKL

SalI and NotI sites were added to cDNA encoding human RANKL residues 140-317 by PCR. The resultant was cloned downstream of Glutathione S-transferase of pGEX-4T-2 (GE healthcare; Genbank Accession Number: U13854) with the use of the endonucleases. Protein expression was induced in BL21 (DE3) Escherichia coli (Invitrogen) with IPTG (final concentration: 0.5 mM). Then, bacterial cells were suspended in an extraction buffer (50 mM Tris-HCl (pH 8.0), 100 mM NaCl, 1 mM EDTA, 1 mM DTT, and 1% (v/v) TritonX-100) and pulverized at 4° C. with the use of a sonicator. After centrifugation at 18000×g for 15 minutes, the supernatant was recovered and applied to a Glutathione Sepharose column. Subsequently, washing with a washing buffer (50 mM Tris-HCl (pH 8.0), 100 mM NaCl, 1 mM DTT, 0.1% (v/v) TritonX-100) was carried out, followed by elution with a Glutathione solution (20 mM reduced glutathione and 50 mM Tris-HCl (pH 8.0)). The molecular weight and purity of purified GST-RANKL were confirmed by SDS-PAGE. The obtained GST-RANKL was subjected to filter filtration. The molecular weight was 47.0 kDa and the purity was 95% or more. In addition, the endotoxin concentration was determined by limulus amebocyte lysate assay and it was confirmed to be less than 1 EU/μg.

### Osteoclast Formation (1)

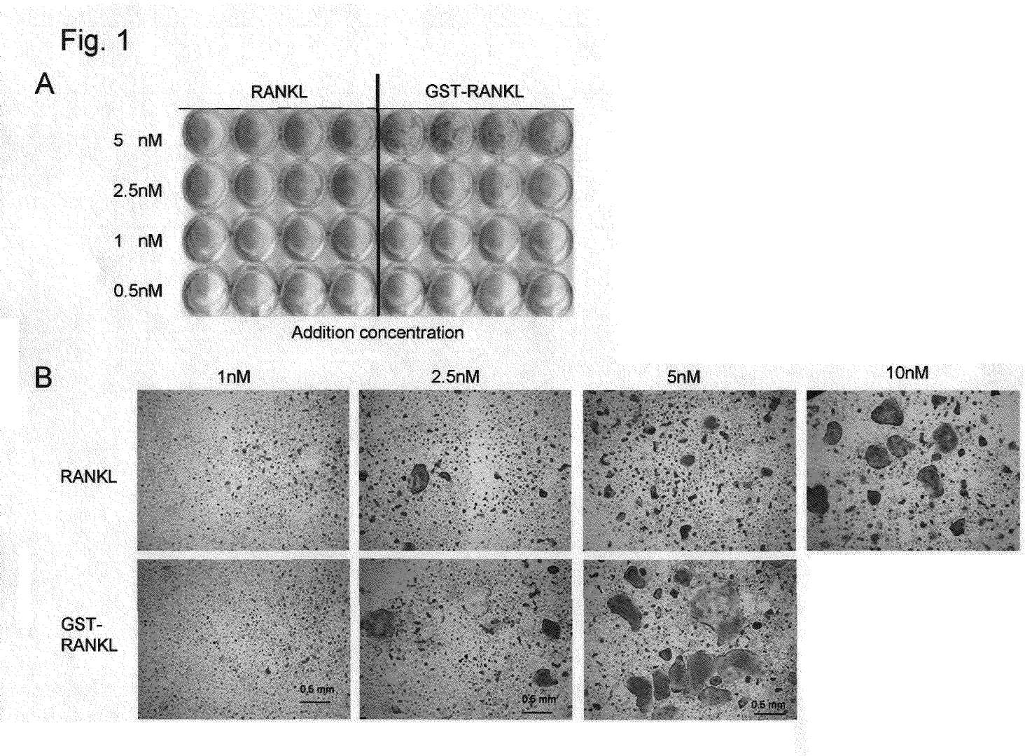

Myelocytes were collected from a 7-week-old ddY mouse and cultured in the presence of M-CSF (10,000 U/mL). Suspension cells were removed and culture was further carried out in the presence of M-CSF for 2 days. Then, the obtained cells were seeded on a 48-well plate at 5×10⁵ cells/well. GST-RANKL and soluble RANKL (Peprotech) were separately added to wells at concentrations of 0.5, 1, 2.5, and 5 nM, followed by culture at 37° C. in a CO₂ incubator. The supernatant of cells that had been cultured for 3 days was discarded and the cells were fixed with a neutral buffered formalin solution for 2 minutes. Acetone/ethanol (acetone:ethanol=1:1) was added thereto for refixation for 1 minute. After fixation, acetone/ethanol was removed, followed by drying. After the addition of 500 μl of a substrate solution (NaPHTOL As-MX Phosphate: 0.26 mM; dimethylformamide: 150 mM; TRAP buffer (CH₃COOH: 30 mM; sodium acetate trihydrate: 85 mM; and sodium tartrate: 25 mM); and Fast Red (SIGMA): 2.33 mM), the cells were confirmed to be stained as a result of microscopic observation, followed by washing with water. Osteoclasts were stained in red in a concentration dependent manner (FIG. 1A). In addition, GST-RANKL-containing cells were found to be stained to a greater extent than soluble RANKL-containing cells. Further, osteoclasts were microscopically observed. Osteoclasts formed from GST-RANKL-containing cells were found to be larger than those formed from soluble RANKL-containing cells. The concentration of soluble RANKL added was increased to 10 nM and examination was conducted in the manner described above. As a result, formation of osteoclasts larger than those obtained with soluble RANKL at 5 nM was confirmed. However, formation of osteoclasts larger than those formed with cells containing GST-RANKL at 5 nM was not observed (FIG. 1B).

GST-RANKL strongly acted on osteoclast formation, and there were clear differences in terms of osteoclast size. Thus, it has been found that addition of GST results in exhibition of strong activity.

### Osteoclast Formation (2)

RAW264 cells were seeded on a 96-well plate (2000 cells/well). GST-RANKL and soluble RANKL (Peprotech) were separately added to wells at concentrations of 0.625, 1.25, and 2.5 nM, followed by culture at 37° C. in a CO₂ incubator. 3 days later, the cells were further cultured for 1 day in a medium containing GST-RANKL or soluble RANKL at an equivalent concentration. In addition, PBS and GST were added to a control case in the same manner as above for comparison. Each measurement value was verified by ANOVA and Dunnett methods.

The supernatant of cultured RAW264 cells was discarded and fixed for 1 minute with the addition of acetone/ethanol (acetone:ethanol=1:1) in a volume of 100 μl per well. After fixation, acetone/ethanol was removed, followed by drying. Incubation was carried out at a room temperature for 30 minutes with the addition of 100 μl of a substrate solution (obtained by adding a 50 mM sodium tartrate solution to a buffer (pH 4.5) at a volume ratio of 1:10 (such buffer containing 1.5 g/ml p-nitrophenyl phosphate and 50 mM citric acid and being obtained by mixing sodium citrate dehydrate (3.3 mM) with citric acid monohydrate (50 mM) so as to adjust the pH to 4.5)).

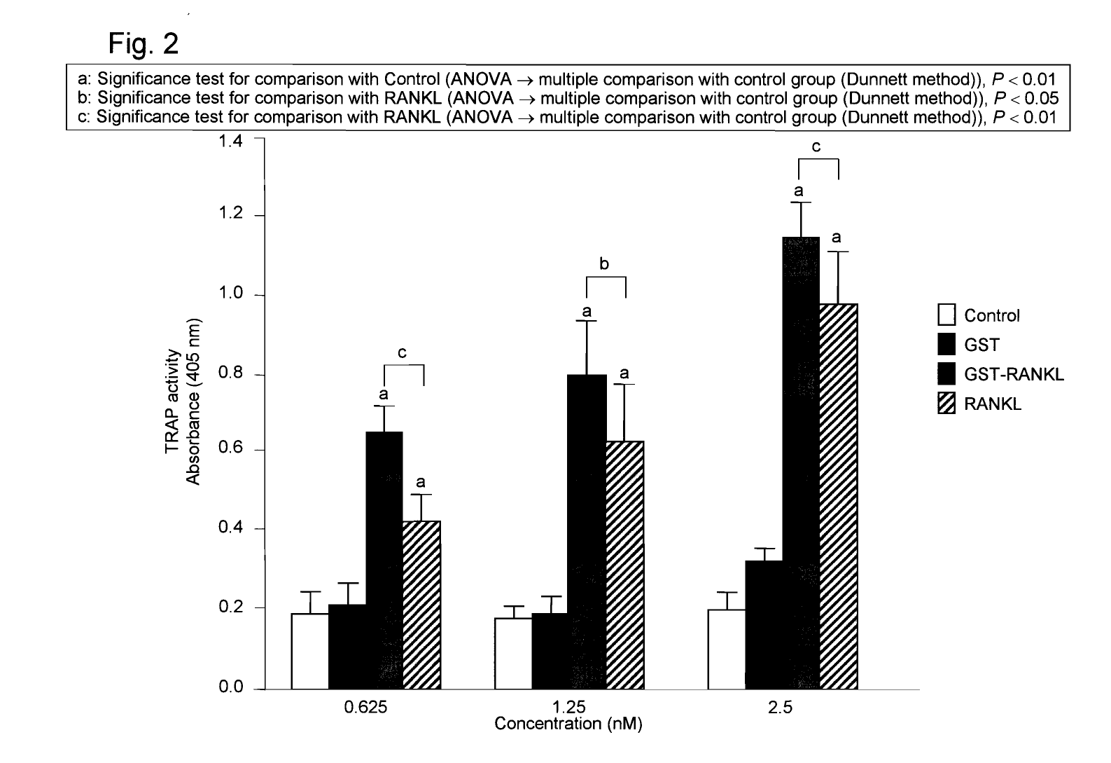

The TRAP activity increased depending on the GST-RANKL and soluble RANKL concentrations. The activity levels of GST-RANKL were significantly higher than those of soluble RANKL at concentrations of 1.25 and 2.5 nM (FIG. 2). At such concentrations, the number of osteoclasts increased in a concentration-dependent manner in both cases of GST-RANKL and soluble RANKL.

### Evaluation of the Life Extension of Matured Osteoclasts and the Bone Resorption Potency of Osteoclasts

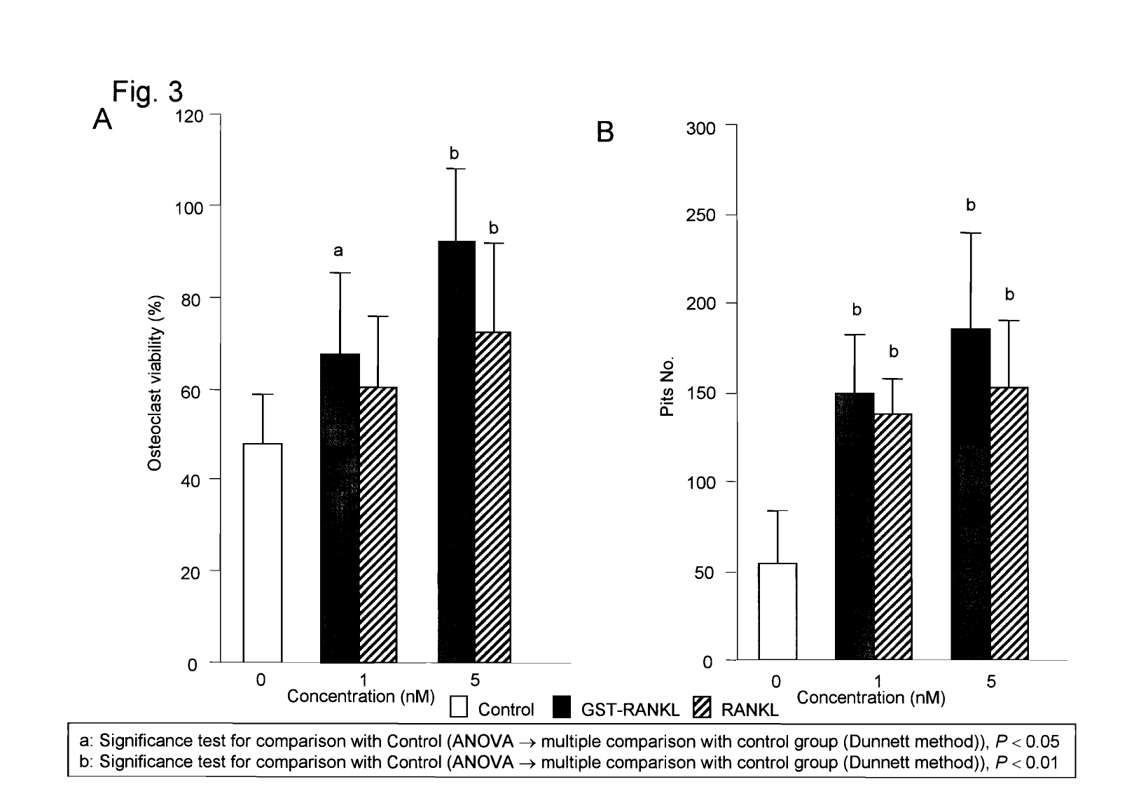

GST-RANKL and soluble RANKL at concentrations of 1 nM and 5 nM were separately added to osteoclasts obtained via coculture of mouse osteoblasts and myelocytes. 0 and 20 hours later, TRAP staining was conducted for the counting of TRAP-positive multinucleated cells. The number of TRAP-positive multinucleated cells determined at 20 hours was divided by the number of cells at 0 hours to calculate the viability (FIG. 3A). The cell viability increased in a concentration-dependent manner with the addition of GST-RANKL and soluble RANKL. In the case of GST-RANKL at 5 nM, the viability was approximately twice as high as that for the control group (p<0.01). In addition, GST-RANKL exhibited stronger effects than soluble RANKL.

All cells, including osteoclasts and osteoblasts obtained via coculture of mouse osteoblasts and myelocytes, were seeded on ivory slices, followed by culture for 2 hours. GST-RANKL and soluble RANKL were separately added thereto, followed by culture for 24 hours. After culture, cells were removed from ivory slices and the pit number was counted by HE staining. The number increased depending on the GST-RANKL and soluble RANKL concentrations, indicating that the both substances promoted bone resorption. However, GST-RANKL exhibited stronger effects than RANKL (FIG. 3B).

### Example 2

### Confirmation of In Vivo Effects of GST-RANKL

GST-RANKL was prepared in the same manner as in Example 1.

### RANKL Administration Test

GST-RANKL or soluble RANKL (Peprotech) was administered 3 times via an intraperitoneal route to groups of 7-week-old female C57BL/6N mice (10 individuals each) at doses of 57 nmol (low dose) and 426 nmol (high dose) every 24 hours. Exsanguination was performed 1.5 hours after the third administration. A group to which PBS was administered in the same manner as above was used as a control group for comparison.

The exsanguinated blood was subjected to measurement of serum bone resorption parameters (calcium, CTx (type I collagen-crosslinked C-peptide telopeptide), TRAP-5b) and serum osteogenesis parameters (osteocalcin and alkaline phosphatase (ALP)). Calcium was measured by the OCPC method (WAKO, 272-21801). CTx (Nordic Bioscience Diagnostics), TRAP-5b (IDS Ltd, SB-TR103), and osteocalcin (Biomedical Technologies Inc.) were measured by ELISA. ALP was measured by the Bessey-Lowry method (WAKO, 274-04401).

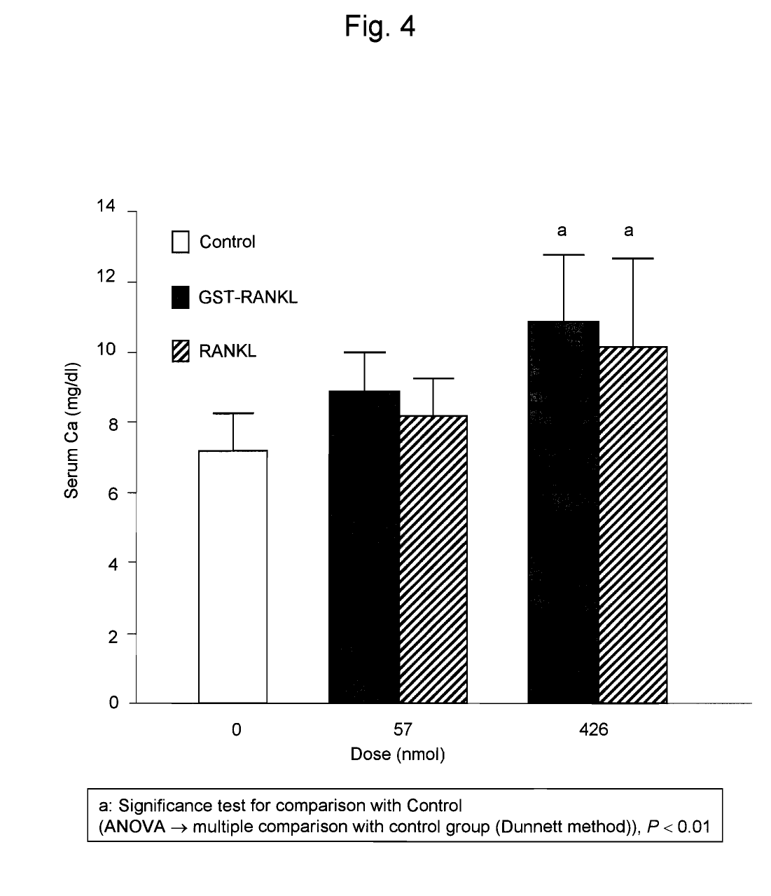

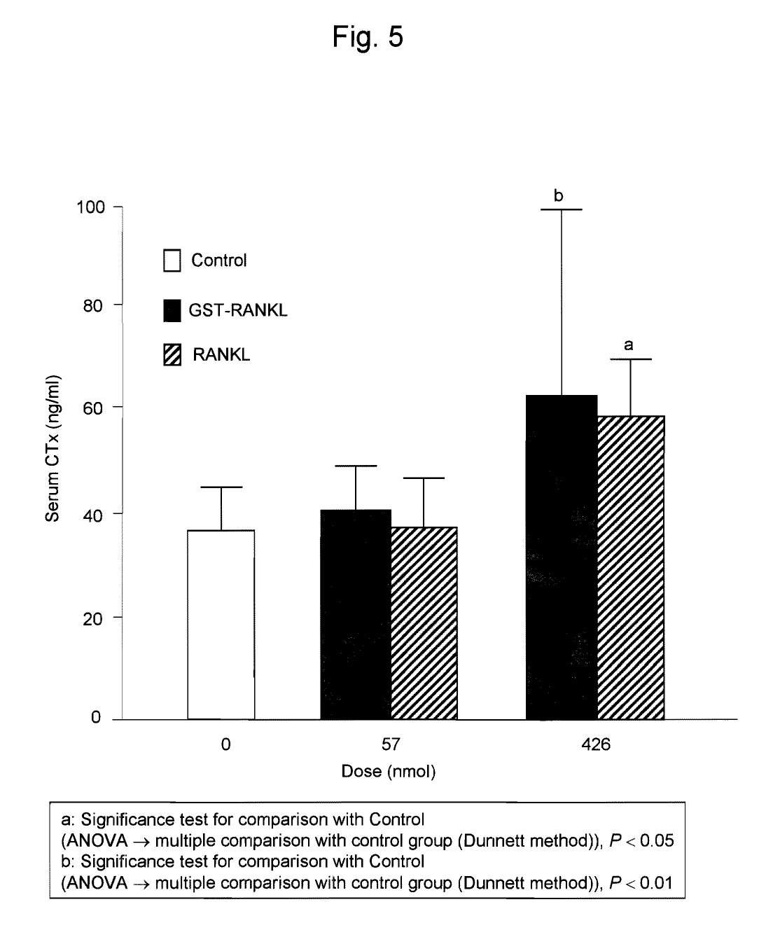

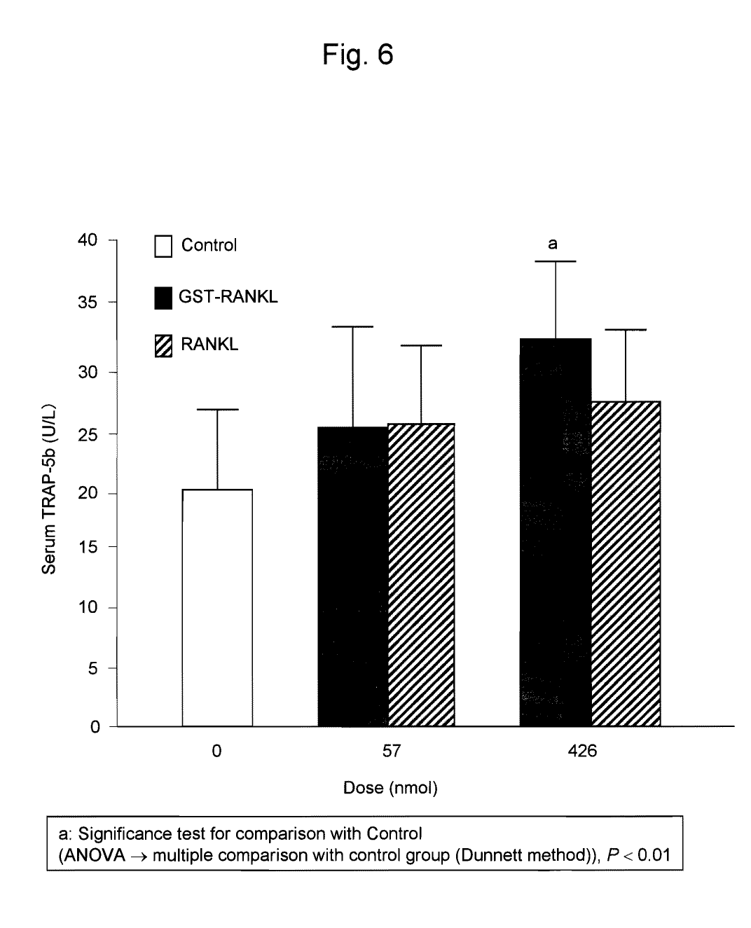

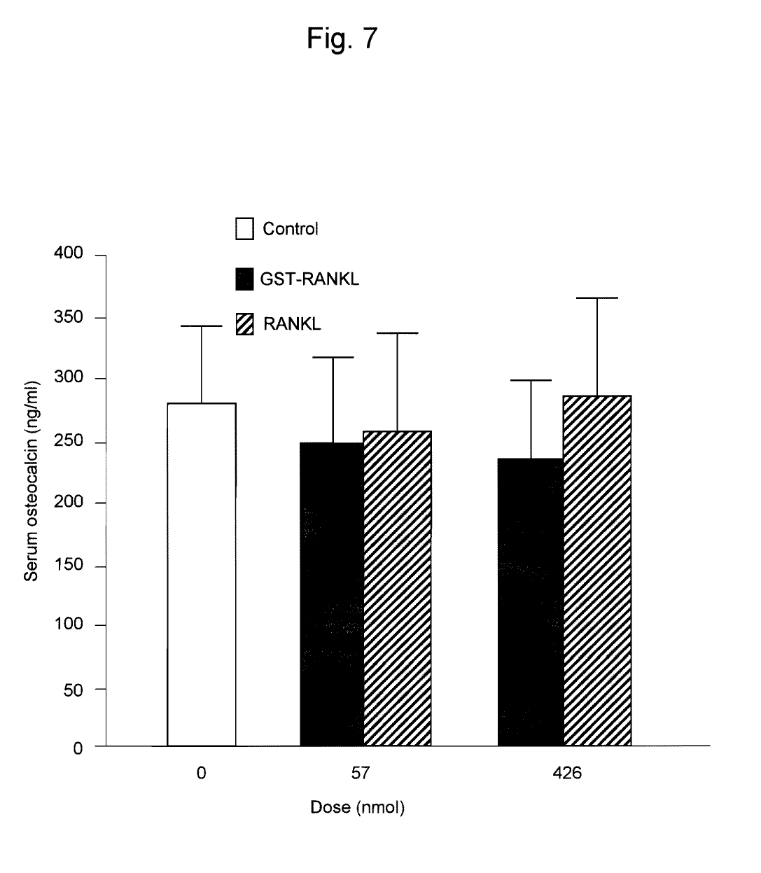

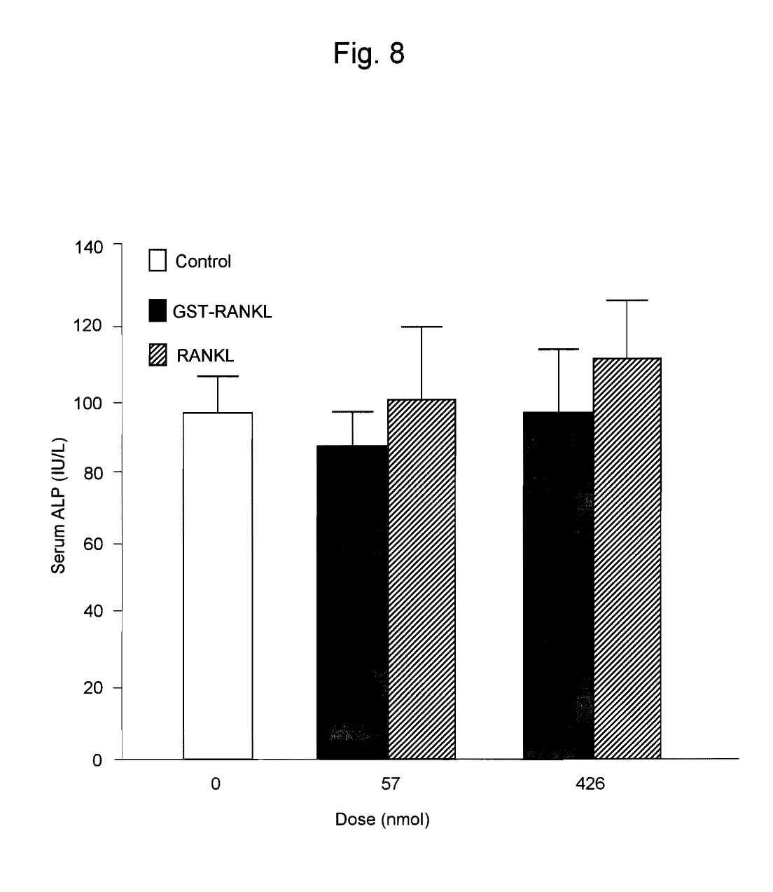

As a result of high-dose administration of either GST-RANKL or soluble RANKL, the serum Ca concentration significantly increased to approximately 1.4-fold as high as that for the control group (p<0.01) (FIG. 4). Also, as a result of high-dose administration, the level of CTx, which is a collagen metabolite, significantly increased to approximately 1.5-fold as high as that for the control group. The significant difference of GST-RANKL was p<0.01 and that of soluble RANKL was p<0.05 (FIG. 5). In addition, as a result of high-dose administration of GST-RANKL, the level of TRAP-5b significantly increased to approximately 1.5-fold as high as that for the control group (p<0.01), while soluble RANKL administration did not result in such significant increase (FIG. 6). Serum osteocalcin and ALP levels did not change after administration of either GST-RANKL or soluble RANKL or high or low doses (FIGS. 7 and 8).

### Bone Density and Bone Morphology Measurement

The following organs were collected from each exsanguinated mouse: the femur, the tibia, the cerebrum, the lungs, the heart, the liver, the thymus, the spleen, kidneys, and the skin. Naturally occurring lesions were observed by HE staining of the cerebrum, the lungs, the heart, the liver, the thymus, the spleen, the kidneys, and the skin.

Regarding the femur, the cancellous bone was subjected to bone density measurement with the use of pQCT at points 0.6 mm, 0.8 mm, and 1.0 mm away from the growth plate on the proximal side of the cancellous bone.

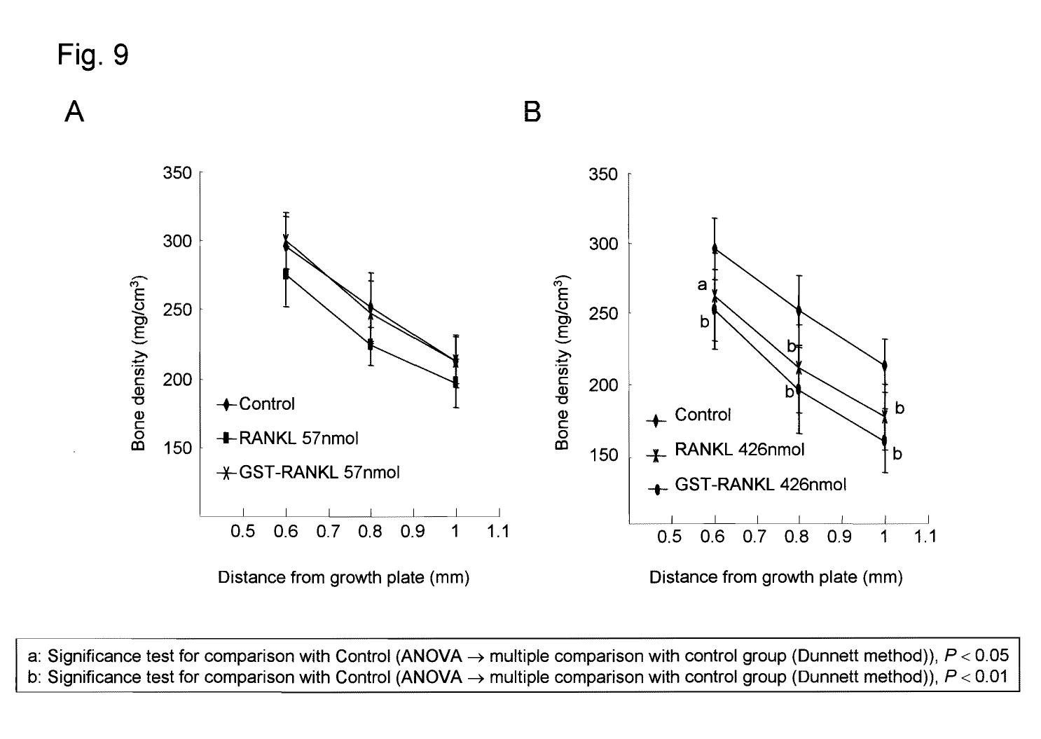

As a result of bone density measurement of the femur with pQCT, the bone density decreased by 10%, 23%, and 30% at the 0.6-, 0.8-, and 1.0-mm points, respectively, in the case of high-dose GST-RANKL administration. Also, the bone density decreased by 10%, 17%, and 20% at the 0.6-, 0.8-, and 1.0-mm points, respectively, in the case of high-dose soluble RANKL administration (FIGS. 9A and 9B). Verification of the significant difference was carried out by the Anova and Dunnett methods. Accordingly, the significant difference was p<0.05 for the point 0.6 mm away from the growth plate in the high-dose soluble RANKL administration group. The significant difference was p<0.01 for the 0.8- and 1.0-mm points in the same group and for each measurement point in the high-dose GST-RANKL administration group (FIG. 9B). In addition, no significant difference was obtained in the low-dose GST-RANKL administration group or the low-dose soluble RANKL administration group (FIG. 9A).

High-dose GST-RANKL and soluble RANKL administration resulted in osteoclast differentiation, life-extending effects on matured osteoclasts, the improvement of the bone resorption potency of osteoclasts, increases in bone resorption parameters, decreases in bone density and bone mass unit, and an increase in the number of osteoclasts. GST-RANKL exhibited stronger activity than that of soluble RANKL in terms of osteoclast formation ability. The results suggested that GST-fused soluble RANKL exhibits improved activity in the phase of osteoclast differentiation, life extension and activation.

### Example 3

### GST-RANKL Activity

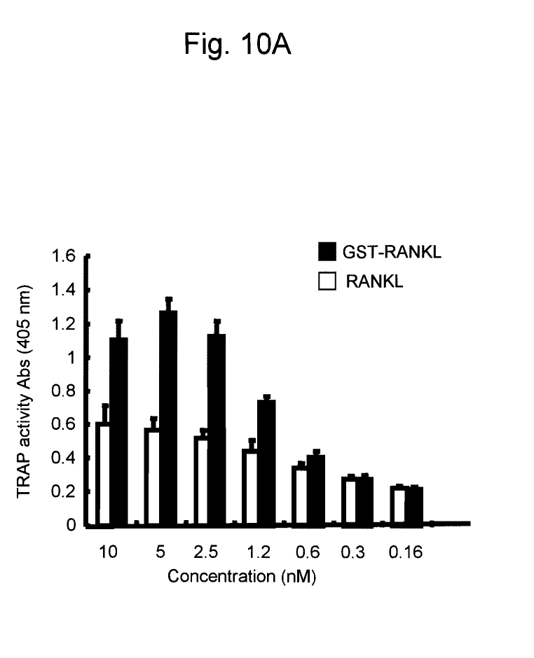

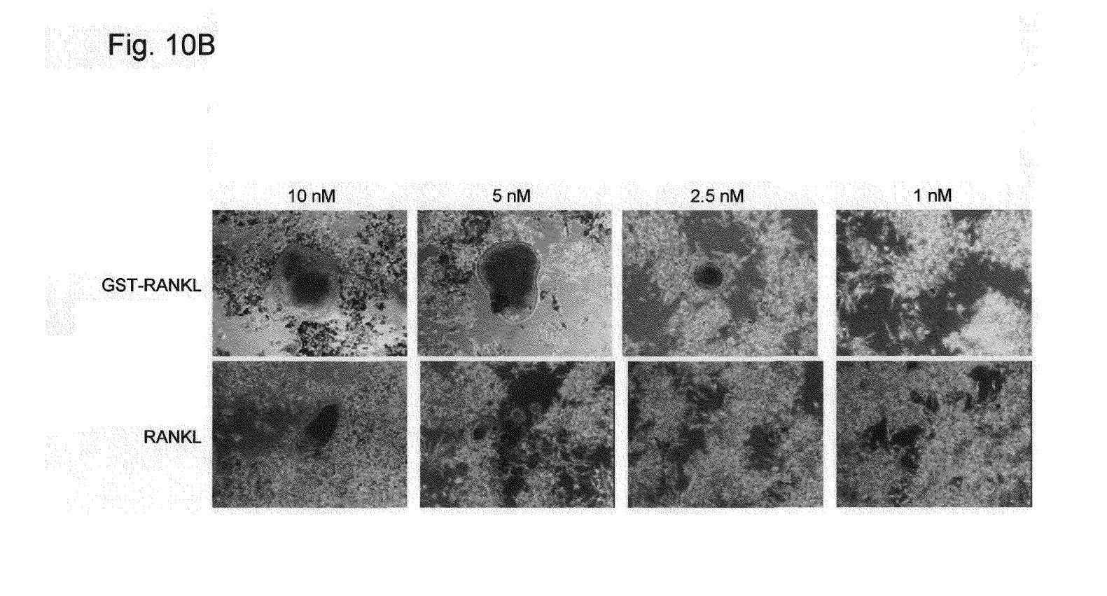

GST-RANKL and soluble RANKL (produced by Peprotech) were separately added to RAW264 cells at serially diluted concentrations of 10, 5, and 2.5 nM. 4 days later, the TRAP activity was determined by the method described in Example 1. The osteoclast differentiation activity after the addition of soluble RANKL (10 nM) was already at the saturation level, and thus the activity did not increase at higher concentrations. As described above, it has been found that soluble RANKL is inferior to GST-RANKL, and that GST-fused RANKL exhibits improved activity and enhanced potency inherent in RANKL such that GST-RANKL exhibits osteoclast differentiation and activation effects to an extent that cannot be achieved with the addition of RANKL alone (FIG. 10A). In addition, osteoclasts in the above case were microscopically observed. Accordingly, GST-RANKL was found to induce osteoclasts larger than those induced by soluble RANKL (FIG. 10B).

### GST-RANKL Preservation Stability

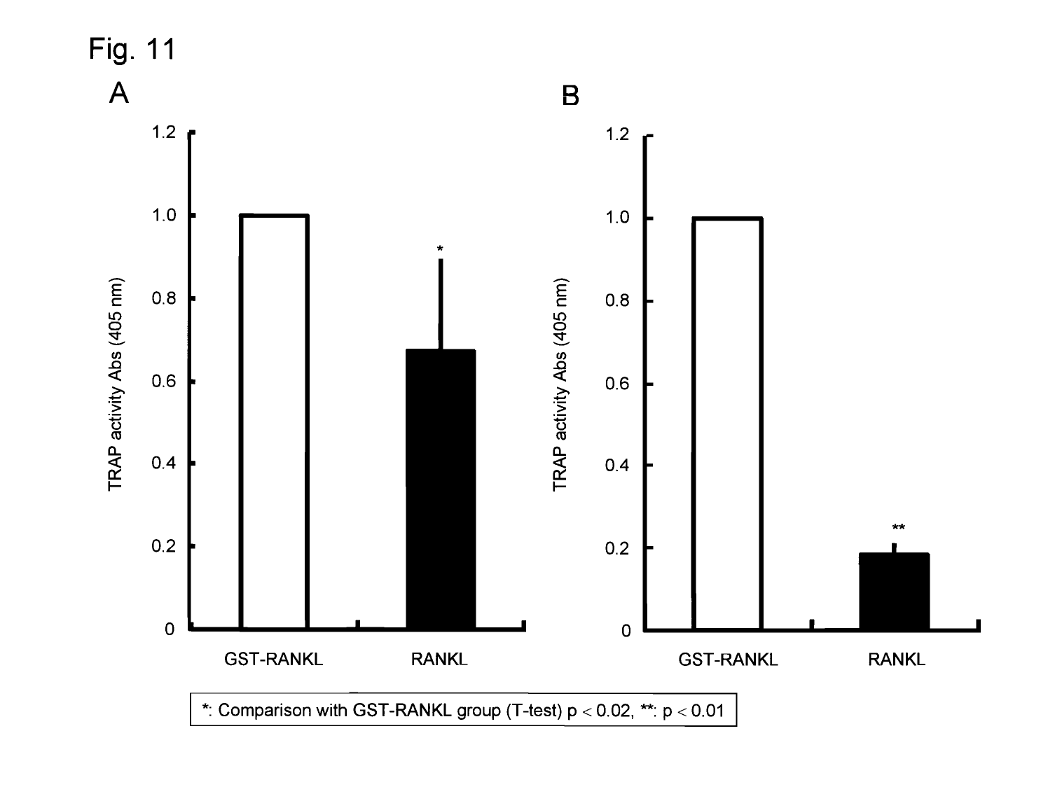

In order to compare the stability of GST-RANKL and that of soluble RANKL, the osteoclast induction activity levels of GST-RANKL and soluble RANKL (produced by Peprotech) (5 nM each) were determined based on the TRAP activity. Thereafter, GST-RANKL and soluble RANKL were preserved at −20° C. or lower for 2 months. Then, the osteoclast induction activity was measured in a similar manner. The obtained measurement results were each represented by the proportion of soluble RANKL activity to GST-RANKL activity, which was determined to be 1, provided that both activity levels were measured at the same time. In an experiment in which soluble RANKL, which is a lyophilized product, was dissolved in a solvent and immediately subjected to measurement, the soluble RANKL activity corresponded to approximately 60% of that of GST-RANKL. However, the soluble RANKL activity obtained 2 months thereafter decreased to a level corresponding to approximately 20% of that of GST-RANKL. The results revealed that preservation stability of GST-fused RANKL was obviously improved (FIGS. 11A and 11B).

### Cross-Reactivity of GST-RANKL

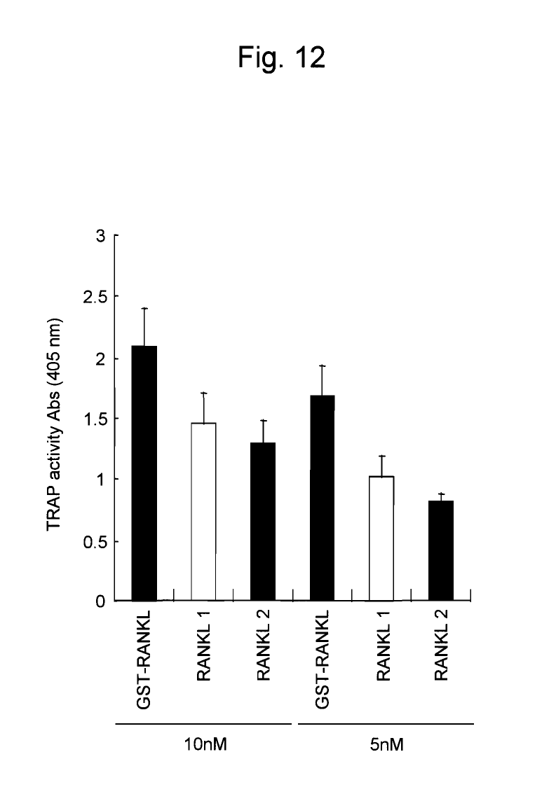

GST-RANKL is one type of human soluble RANKL. However, as a result of comparison with mouse soluble RANKLs (Peprotech and R&D) in terms of osteoclast induction activity (at concentrations of 10 nM and 5 nM), GST-RANKL was found to have activity at least twice as high as that of mouse soluble RANKL, despite the fact that RAW264 cells are mouse-derived cells (FIG. 12). The results revealed that GST-fused human soluble RANKL (GST-RANKL) exhibits stronger osteoclast induction activity in RAW264 cells (having mouse RANKs serving as RANKL receptors) than mouse soluble RANKL. The results also indicate that GST-fused human soluble RANKL acts on mouse RANK in a way that transcends species differences, and that the effects thereof are stronger than those obtained by the mouse ligand-receptor reaction between mouse soluble RANKL and mouse RANK.

### Example 4

### Osteoclast Differentiation

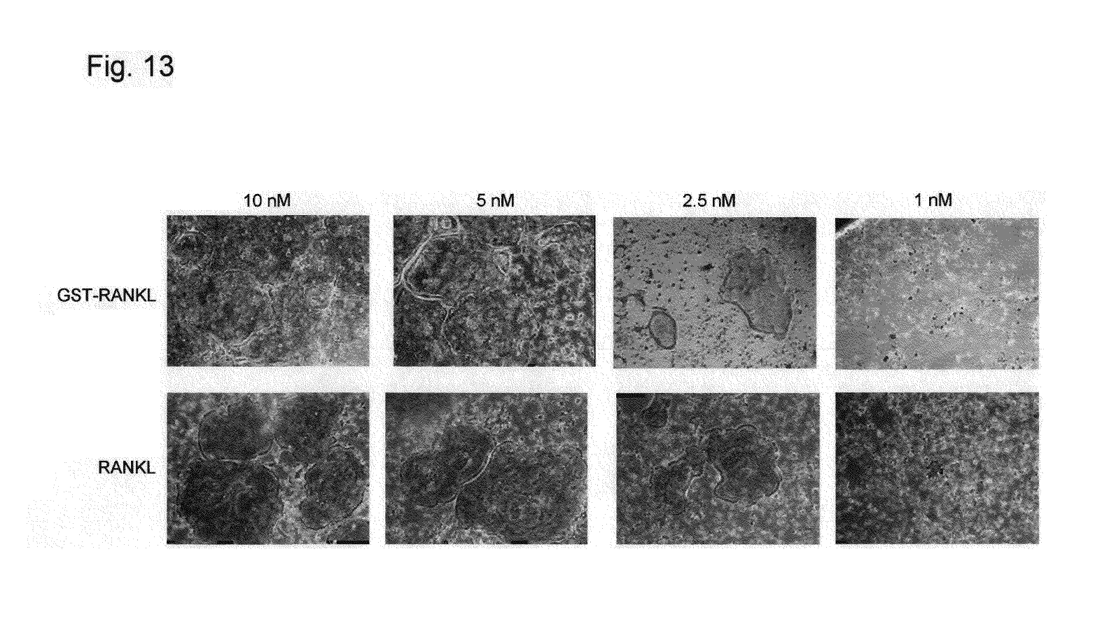

Myelocytes were collected from a 7-week-old ddY mouse and cultured in the presence of M-CSF (10,000 U/mL). Suspension cells were removed and culture was further carried out in the presence of M-CSF for 2 days. Then, the cells were seeded on a 48-well plate at 5×10⁵ cells/well. GST-RANKL and soluble RANKL (Peprotech) were separately added to wells at concentrations of 1, 2.5, 5, and 10 nM, followed by culture at 37° C. in a CO₂ incubator. The supernatant of cells that had been cultured for 3 days was discarded and the cells were fixed with a neutral buffered formalin solution for 2 minutes. Acetone/ethanol (acetone:ethanol=1:1) was added thereto for refixation for 1 minute. After fixation, acetone/ethanol was removed, followed by drying. After the addition of 500 μl of a substrate solution (NaPHTOL As-MX Phosphate: 0.26 mM; dimethylformamide: 150 mM; TRAP buffer (CH₃COOH: 30 mM; sodium acetate trihydrate: 85 mM; and sodium tartrate: 25 mM); and Fast Red (SIGMA): 2.33 mM), the cells were confirmed to be stained as a result of microscopic observation, followed by washing with water. Osteoclasts were stained in red in a concentration dependent manner (FIG. 13). In addition, GST-RANKL-containing cells were found to be stained more than soluble RANKL-containing cells. Further, osteoclasts were microscopically observed. Osteoclasts formed from GST-RANKL-containing cells were found to be larger than those formed from soluble RANKL-containing cells (FIG. 13).

GST-RANKL strongly acted on osteoclast formation and there were clear differences in terms of osteoclast size. Thus, it has been found that addition of GST results in exhibition of strong activity.

### INDUSTRIAL APPLICABILITY

Production of a fused protein of RANKL with an epitope tag, such as GST-RANKL, results not only in the improvement of the specific activity of soluble RANKL but also in the enhancement of the potency thereof. That is, in an in vitro osteoclast formation system, an RANKL fused protein such as GST-RANKL can cause formation of a greater number of much larger osteoclasts than those formed by increasing the soluble RANKL concentration to the saturation level. This indicates that the potency of such fused protein is improved in terms of the number of osteoclasts that can be formed and activated, in addition to the specific activity that can be increased. Accordingly, it has become possible to allow RANKL to exhibit strong activity not only in vitro but also in vivo. A fused protein of RANKL with an epitope tag can be preferably used as a reagent used for bone metabolism studies. Further, when it is administered in vivo to an animal, the bone density and the bone mass of the animal decrease. Therefore, the fused protein can be used for production of osteopenia animal models. Further, the activity of RANKL is not limited to osteoclast differentiation and activation, and it also includes an activity of promoting lymphocyte differentiation, dendritic cell activation, mammary gland epithelial cell differentiation, lymph node formation, and the like.

A fused protein of RANKL with an epitope tag can be preferably used as a reagent used for bone metabolism studies. Further, when it is administered in vivo to an animal, the bone density and the bone mass of the animal decrease. Therefore, the fused protein can be used for production of osteopenia animal models.

Further, the activity of RANKL is not limited to osteoclast differentiation and activation, and it also includes an activity of promoting lymphocyte differentiation, dendritic cell activation, mammary gland epithelial cell differentiation, lymph node formation, and the like. Therefore, the present invention also encompasses the use of the above fused protein as a reagent capable of inducing promotion of such effects.

All publications, patents, and patent applications cited herein are incorporated herein by reference in their entirety.

### Free Text of Sequence Listing

SEQ ID NOS: 3 to 18 (synthesized)

1. An agent for differentiating and activating osteoclasts, containing, as an active ingredient, a fused protein of soluble RANKL with an epitope tag in which the differentiating and activating function of soluble RANKL is improved.

2. The agent for differentiating and activating osteoclasts according to claim 1, which is used in vitro.

3. The agent for differentiating and activating osteoclasts according to claim 1, which is used in vivo.

4. The agent for differentiating and activating osteoclasts according to claim 1, which has improved preservation stability.

5. The agent for differentiating and activating osteoclasts according to claim 1, which has interspecies cross-reactivity.

6. An agent for inducing osteopenia in an animal, containing, as an active ingredient, a fused protein of soluble RANKL with an epitope tag in which s the differentiating and activating function of soluble RANKL is improved.

7. The agent for inducing osteopenia in an animal according to claim 6, which has interspecies cross-reactivity.

8. An agent for producing an osteopenia animal model, containing, as an active ingredient, a fused protein of soluble RANKL with an epitope tag in which the differentiating and activating function of soluble RANKL is improved.

9. The agent for producing an osteopenia animal model according to claim 8, which has interspecies cross-reactivity.

10. The agent for differentiating and activating osteoclasts according to claim 1, wherein the epitope tag is glutathione-S-transferase.

11. The agent for inducing osteopenia in an animal according to claim 6, wherein the epitope tag is glutathione-S-transferase.

12. The agent for producing an osteopenia animal model according to claim 8, wherein the epitope tag is glutathione-S-transferase.

13. A method of differentiating and forming osteoclasts from myelocytes, spleen cells, or peripheral blood cells, comprising culturing myelocytes, spleen cells, or peripheral blood cells collected from an animal in the presence of the agent for differentiating and activating osteoclasts according to claim 1.

14. A method of inducing osteopenia in a non-human animal, comprising administering the agent for differentiating and activating osteoclasts according to claim 1 to the non-human animal.

15. A composition selected from the group consisting of an agent for differentiating lymphocytes, a dendritic cell activator, an agent for differentiating mammary gland epithelial cells, and an agent for forming lymph nodes, which contains, as an active ingredient, a fused protein of soluble RANKL with an epitope tag in which at least one function of soluble RANKL selected from the group consisting of a lymphocyte differentiating function, a dendritic cell activating function, a mammary gland epithelial cell differentiating function, and a lymph node forming function has been improved.

16. The composition according to claim 15, which has interspecies cross-reactivity.

17. The composition according to claim 15, wherein the epitope tag is glutathione-S-transferase.


SEQUENCE LISTING (24 sequences)

SEQ ID NO: 1 - DNA, 2201 bp, Homo sapiens
  CDS (129)..(1082)
ggccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacctgccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatttgtcaagagtagttatataattgttgaacaggtgtttttccacaagtgccgcaaattgtacctttttttttttttcaaaatagaaaagttattagtggtttatcagcaaaaaagtccaattttaatttagtaaatgttattttatactgtacaataaaaacattgcctttgaatgttaattttttggtacaaaaataaatttatatgaaaaaaaaaaaaaaaaaaaaaaaaaaaaaaaa

SEQ ID NO: 2 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 3 - protein, 8 aa, Artificial
  Synthetic
DYKDDDDK

SEQ ID NO: 4 - protein, 10 aa, Artificial
  Synthetic
EQKLISEEDL

SEQ ID NO: 5 - protein, 14 aa, Artificial
  Synthetic
GKPIPNPLLGLDST

SEQ ID NO: 6 - protein, 6 aa, Artificial
  Synthetic
HQHQHQ

SEQ ID NO: 7 - protein, 9 aa, Artificial
  Synthetic
YPYDVPDYA

SEQ ID NO: 8 - protein, 6 aa, Artificial
  Synthetic
DTYRYI

SEQ ID NO: 9 - protein, 11 aa, Artificial
  Synthetic
MASMTGGQQMG

SEQ ID NO: 10 - protein, 11 aa, Artificial
  Synthetic
YTDIEMNRLGK

SEQ ID NO: 11 - protein, 5 aa, Artificial
  Synthetic
DDDDK

SEQ ID NO: 12 - protein, 16 aa, Artificial
  Synthetic
KETAAAKFERQHIDSC

SEQ ID NO: 13 - protein, 12 aa, Artificial
  Synthetic
MKAEFRRQESDR

SEQ ID NO: 14 - protein, 12 aa, Artificial
  Synthetic
MRDALDRLDRLA

SEQ ID NO: 15 - protein, 12 aa, Artificial
  Synthetic
MKDGEEYSRAFR

SEQ ID NO: 16 - protein, 9 aa, Artificial
  Synthetic
EEEEYMPME

SEQ ID NO: 17 - protein, 12 aa, Artificial
  Synthetic
CTPTDVPDYASL

SEQ ID NO: 18 - protein, 6 aa, Artificial
  Synthetic
PPEPET

SEQ ID NO: 19 - DNA, 1275 bp, Artificial
  GST-RANKL (aa127-317)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccaggaattcccgggtcgactgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattga

SEQ ID NO: 20 - protein, 442 aa, Artificial
  GST-RANKL (aa127-317)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPGIPGSTRAAASLVPRGSPGIPGSTVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 21 - DNA, 1236 bp, Artificial
  GST-RANKL (aa140-317)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccaggaattcccgggtcgactatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattga

SEQ ID NO: 22 - protein, 429 aa, Artificial
  GST-RANKL (aa140-317)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPGIPGSTRAAASLVPRGSPGIPGSTIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 23 - DNA, 1179 bp, Artificial
  GST-RANKL (aa159-317)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccaggaattcccgggtcgactaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattga

SEQ ID NO: 24 - protein, 410 aa, Artificial
  GST-RANKL (aa159-317)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPGIPGSTRAAASLVPRGSPGIPGSTKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID